(19) United States
(12) Patent Application Publication
(10) Pub. No.: US 2026/0324290 A1
(43) Pub. Date: Oct. 15, 2026

(54) TEXTURED LEAD-BASED RELAXOR FERROELECTRIC MATERIALS FOR MEDICAL APPLICATIONS AND METHODS FOR FABRICATING LEAD-BASED RELAXOR FERROELECTRIC MATERIALS USING TEMPLATED GRAIN GROWTH METHODS AND LEAD-BASED RELAXOR FERROELECTRIC TEMPLATES

(71) Applicant: GE Precision Healthcare LLC, Waukesha, WI (US)

(72) Inventor: Jeremie Barrel, Opio (FR)

(21) Appl. No.: 19/178,361

(22) Filed: Apr. 14, 2025

(51) Int. Cl. H10N 30/85; A61B 8/00; A61L 31/02; B06B 1/06; C30B 29/30; C30B 29/32; H10N 30/092

(52) U.S. Cl. CPC H10N 30/852; A61B 8/4483; A61L 31/022; B06B 1/0622; C30B 29/30; C30B 29/32; H10N 30/092; A61B 2562/12; B06B 2201/76

(57) ABSTRACT

A textured lead-based relaxor ferroelectric material for a transducer element of an ultrasound probe may include matrix particles formed of a textured lead-based relaxor ferroelectric material, and template particles formed of the textured lead-based relaxor ferroelectric material. A method of fabricating a textured lead-based relaxor ferroelectric material for a transducer element of an ultrasound probe may include fabricating a single-crystal lead-based relaxor ferroelectric material, fabricating template particles using the single-crystal lead-based relaxor ferroelectric material, and fabricating a textured lead-based relaxor ferroelectric material using a templated grain growth method, the template particles, and matrix particles of lead-based relaxor ferroelectric material having a same material composition as the template particles.

### TECHNICAL FIELD

The present disclosure relates to textured lead-based relaxor ferroelectric materials for medical applications. Further, the present disclosure relates to methods for fabricating textured lead-based relaxor ferroelectric materials for medical applications using templated grain growth methods, a single-crystal formed of a lead-based relaxor ferroelectric material, matrix particles formed of the lead-based relaxor ferroelectric material, and template particles formed of the lead-based relaxor ferroelectric material. Further still, the present disclosure relates to a transducer element of an ultrasound probe that is formed of a textured lead-based relaxor ferroelectric material that is fabricated using templated grain growth methods and matrix particles and template particles formed of a same lead-based relaxor ferroelectric material. Further still, the present disclosure relates to an ultrasound probe having one or more transducer elements formed of textured lead-based relaxor ferroelectric materials that are fabricated using templated grain growth methods and matrix particles and template particles formed of a same lead-based relaxor ferroelectric material.

### BACKGROUND

An ultrasound probe may include transducer elements formed of piezoelectric materials for various medical applications, such as ultrasound imaging, ultrasound measurement, tissue ablation, ultrasound diagnosis, therapeutic delivery, or the like. The piezoelectric materials may deform in response to an electrical signal in order to generate an ultrasound signal, and may deform in response to an echo signal in order to generate an electrical signal.

Single-crystal lead-based relaxor ferroelectric materials (e.g., lead magnesium niobate-lead titanate (Pb(Mg_{1/3}Nb_{2/3})O₃-PbTiO₃ (“PMN-PT”)), lead indium niobate-lead magnesium niobate-lead titanate ((Pb(In_{1/2}Nb_{1/2})O₃- Pb(Mg_{1/3}Nb_{2/3})O₃-PbTiO₃ (“PIN-PMN-PT”), or the like) may be used for the transducer elements because of the relatively greater performance (e.g., greater piezoelectric charge coefficient, greater electromechanical coupling factor, etc.) as compared to lead zirconate titanate (Pb(ZrTi) (“PZT”)) materials.

The Bridgman method for growing single crystals of lead-based relaxor ferroelectric materials which is used in productions worldwide is expensive due to the use of platinum crucibles and difficult due to the melting occurrence.

Textured lead-based relaxor ferroelectric ceramics may be fabricated using a templated grain growth method. A templated grain growth method includes, among other things, adding Single Crystal template particles to matrix particles formed of lead-based relaxor ferroelectric ceramics, orienting the template particles within the matrix particles formed of lead-based relaxor ferroelectric material, and sintering the matrix particles formed of the lead-based relaxor ferroelectric material and the template particles to fabricate a textured lead-based relaxor ferroelectric ceramics .Textured lead-based relaxor ferroelectric ceramics offer a balance between the high performance of single-crystals formed of lead-based relaxor ferroelectric materials and the cost-effectiveness of regular piezoelectric materials. In this way, textured lead-based relaxor ferroelectric materials provide improved piezoelectric properties at a fraction of the cost of single-crystals formed of lead-based relaxor ferroelectric materials.

Textured lead-based relaxor ferroelectric materials may be developed for use for, among other things, sonar applications, nondestructive testing applications, and actuator applications. For the foregoing applications, the textured lead-based relaxor ferroelectric materials are generally fabricated using barium titanate (BaTiO₃) material for the template particles. The size of the ceramic grains of the template particles formed of barium titanate is not particularly detrimental in the fabrication of, and the resultant properties of, transducers for the foregoing applications. However, barium titanate template particles are not especially suitable for medical applications involving, for example, ultrasound probes. For instance, barium titanate materials possess lower piezoelectric properties than lead-based relaxor ferroelectric materials, which increases the volume ratio of barium titanate template particles in the matrix particles of lead-based relaxor ferroelectric material and which reduces the piezoelectric properties of the resultant textured lead-based relaxor ferroelectric materials.

The number of barium titanate template particles in the matrix particles formed of lead-based relaxor ferroelectric material is generally limited, which necessitates a large grain of lead-based relaxor ferroelectric material to be grown. The degree of orientation in a textured lead-based relaxor ferroelectric material decreases with an increasing number of grains because crossing grain boundaries degrades the degree of orientation. Large grain growth makes achieving a high lotgering factor easier as it reduces the number of grains between templates. Moreover, barium titanate template particles as large as 30-40 µm are generally used. The grain size of traditional piezoelectric materials used in ultrasound probes are about 2-4 µm, which may be appropriate for a transducer element size of 100 µm thickness and 250 µm width including subdicing of 30 µm. Barium titanate template particles would create dicing and homogeneity issues for transducers for medical applications.

### SUMMARY

This summary introduces concepts that are described in more detail in the detailed description. It should not be used to identify essential features of the claimed subject matter, nor to limit the scope of the claimed subject matter.

In an aspect, a textured lead-based relaxor ferroelectric material for a transducer element of an ultrasound probe may include matrix particles formed of a lead-based relaxor ferroelectric material; and template particles formed of the lead-based relaxor ferroelectric material.

In another aspect, a method of fabricating a textured lead-based relaxor ferroelectric material for a transducer element of an ultrasound probe may include fabricating a single-crystal formed of a lead-based relaxor ferroelectric material; fabricating template particles using the single-crystal formed of the lead-based relaxor ferroelectric material; and fabricating the textured lead-based relaxor ferroelectric material using a templated grain growth method, matrix particles formed of the lead-based relaxor ferroelectric material, and the template particles formed of the lead-based relaxor ferroelectric material.

In yet another aspect, a textured lead-based relaxor ferroelectric material for a transducer element of an ultrasound probe may be fabricated by a method comprising: fabricating a single-crystal formed of a lead-based relaxor ferroelectric material; fabricating template particles using the single-crystal formed of the lead-based relaxor ferroelectric material; and fabricating the textured lead-based relaxor ferroelectric material using a templated grain growth method, matrix particles formed of the lead-based relaxor ferroelectric material, and the template particles formed of the lead-based relaxor ferroelectric material.

### BRIEF DESCRIPTION OF DRAWINGS

FIG. 1 is a diagram of example components of an ultrasound system including one or more transducer elements formed of a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material.

FIG. 2 is a diagram of an example ultrasound probe of the ultrasound system including one or more transducer elements formed of a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material.

FIG. 3 is a flowchart of an example process for fabricating a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material.

FIGS. 4A-4D are diagrams of a process for fabricating a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material.

FIG. 5 is a diagram of a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material.

FIG. 6 is a diagram of a textured lead-based relaxor ferroelectric material fabricated using a templated grain growth method, matrix particles formed of a lead-based relaxor ferroelectric material, and template particles formed of barium titanate material.

FIG. 7 is a flowchart of an example process for fabricating a transducer element of an ultrasound probe using a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material.

### DETAILED DESCRIPTION

As addressed above, textured lead-based relaxor ferroelectric materials offer a balance between the high performance of single-crystal piezoelectric materials and the cost-effectiveness of regular piezoelectric materials. However, as further addressed above, the fabrication of textured lead-based relaxor ferroelectric materials using barium titanate templates is not suitable for medical applications using ultrasound probes because of the relatively large size of barium titanate templates and the reduced piezoelectric properties of barium titanate material.

Some embodiments herein provide for the fabrication of a textured lead-based relaxor ferroelectric material that is fabricated using a templated grain growth method, a single-crystal formed of a lead-based relaxor ferroelectric material, matrix particles formed of the lead-based relaxor ferroelectric material, and template particles formed of the lead-based relaxor ferroelectric material. In this way, the single-crystal, the template particles, and the matrix particles are all formed of the same underlying material. Some embodiments herein use template particles formed of lead-based relaxor ferroelectric materials that are smaller than template particles formed of barium titanate and that have the same material composition as the matrix particles formed of the lead-based relaxor ferroelectric material, which improves orientation during grain growth and increases the lotgering factor of the fabricated textured lead-based relaxor ferroelectric materials. The embodiments herein enable the incorporation of a greater number of template particles in the matrix particles for templated grain growth because the template particles are formed of the same underlying material as the material of the matrix particles. Increasing the number of template particles allows for a reduction in the textured ceramic grain size while still achieving similar crystal orientation degree, or lotgering factor, because the distance between two template particles is reduced.

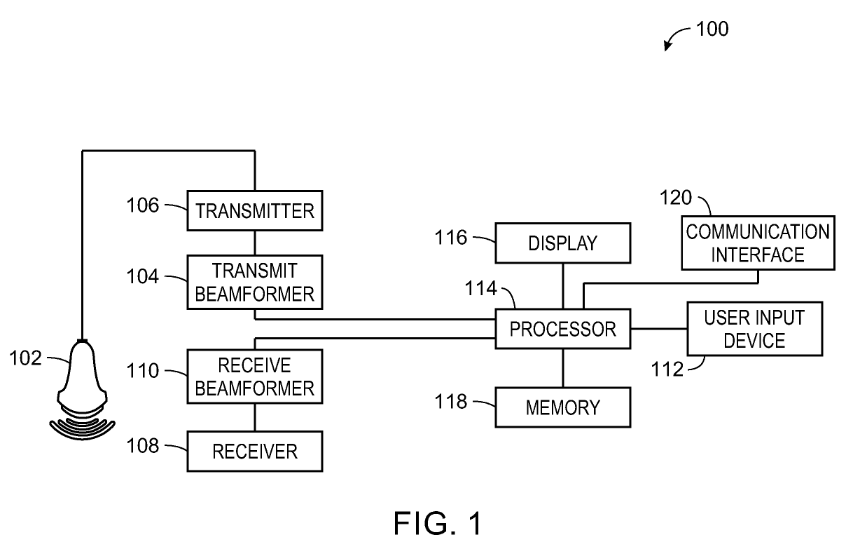

FIG. 1 is a diagram of example components of an ultrasound system 100 including one or more transducer elements 206 formed of a textured lead-based relaxor ferroelectric material fabricated using a single-crystal formed of a lead-based relaxor ferroelectric material, matrix particles formed of the lead-based relaxor ferroelectric material, and template particles formed of the lead-based relaxor ferroelectric material. As shown in FIG. 1, the ultrasound system 100 may include an ultrasound probe 102, a transmit beamformer 104, a transmitter 106, a receiver 108, a receive beamformer 110, a user input device 112, a processor 114, a display 116, a memory 118, and a communication interface 120. The foregoing components may be connected via wired or wireless connections.

The ultrasound probe 102 may be configured to acquire ultrasound data. For example, the ultrasound probe 102 may be a linear probe, a phased array probe, a curved linear probe coupled with a position tracking system, a mechanically steered linear array transducer, a phased array transducer, a curved linear array transducer, an electronically steered 2D transducer array, an electronic 3D (e3D) probe, an electronic 4d (e4D) probe, a low profile wearable patch version of any of the foregoing probes, or the like. According to an embodiment, the ultrasound probe 102 may be configured to generate ultrasound signals, emit the ultrasound signals towards the region of interest of a subject, receive echo ultrasound signals that are back-scattered from the region of interest of the subject, generate ultrasound data based on the echo ultrasound signals, and output the ultrasound data.

The transmit beamformer 104 may be configured to apply delay times to electrical signals provided to elements of the ultrasound probe 102 to focus corresponding ultrasound signals at the region of interest. The transmitter 106 may be configured to transmit electrical signals to the elements of the ultrasound probe 102 to drive the elements of the ultrasound probe 102 to emit ultrasound signals towards the region of interest. The elements of the ultrasound probe 102 may be configured to receive the electrical signals from the transmitter 106, convert the electrical signals into ultrasound signals, and emit the ultrasound signals towards the region of interest. The elements of the ultrasound probe 102 may be configured to receive echo ultrasound signals that are back-scattered by the region of interest, convert the echo ultrasound signals into electrical signals, and provide the electrical signals to the receiver 108. The receiver 108 may be configured to receive electrical signals from the elements, and provide the electrical signals to the receive beamformer 110. The receive beamformer 110 may apply delay times to the electrical signals received from the elements of the ultrasound probe 102.

The user input device 112 may be configured to receive a user input, and provide the user input to the processor 114. The user input device 112 may be a user interface. For example, the user input device 112 may be a touch screen display, a keyboard, a keypad, a mouse, a button, a switch, a microphone, or the like. Additionally, or alternatively, the user input device 112 may be configured to sense information. For example, the user input device 112 may sense information from an electro-magnetic positioning system, an inertial measurement system, an accelerometer, a gyroscope, an actuator, or the like.

The processor 114 may be configured to control the ultrasound system 100. For example, the processor 114 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. The processor 114 may be implemented in hardware, firmware, or a combination of hardware and software. The processor 114 may include one or more processors 114 configured to control the ultrasound system 100. For example, a single processor 114 may be configured to control all of the operations of the ultrasound system 100. Alternatively, multiple processors 114, collectively, may be configured to control the ultrasound system 100, and each of the multiple processors 114 may be configured to perform a subset of the operations associated with controlling the ultrasound system 100. For example, a first processor 114 may perform a first subset of the operations associated with controlling the ultrasound system 100, a second processor 114 may be configured to perform a second subset of the operations associated with controlling the ultrasound system 100, etc.

The processor 114 may be configured to control the ultrasound probe 102 to acquire ultrasound data. The processor 114 may be configured to control which of the elements of the ultrasound probe 102 are active, and control the shape of a beam emitted from the ultrasound probe 102. The processor 114 may generate ultrasound images for display. For example, the processor 114 may generate B-mode images, color Doppler images, M-mode images, color M-mode images, or the like. The ultrasound images may be 3D images, 2D images, single plane images, bi-plane images, three-plane images, multi-plane images, or the like. The ultrasound images may correspond to various anatomical planes (e.g., sagittal, coronal, and transverse) of the region of interest.

The display 116 may be configured to display information. For example, the display 116 may be a monitor, an LED display, a cathode ray tube, a projector display, a touchscreen, tablet computer, mobile phone, or the like. The display 116 may display ultrasound images based on the ultrasound data in real-time. For example, the display 116 may display the ultrasound images within one second, two seconds, five seconds, etc., of the ultrasound data being acquired by the ultrasound probe 102.

The memory 118 may be configured to store information and/or instructions for use by the processor 114. The memory 118 may be a non-transitory computer-readable medium. For example, the memory 118 may be a random access memory (RAM), a read only memory (ROM), and/or another type of dynamic or static storage device (e.g., a flash memory, a magnetic memory, and/or an optical memory) that stores information and/or instructions for use by the processor 114. The memory 118 may be configured to store instructions that, when executed by the processor 114, cause the processor 114 to perform the operations described herein.

The communication interface 120 may be configured to enable the processor 114 to communicate with other systems, such as via a wired connection, a wireless connection, or a combination of wired and wireless connections. For example, the communication interface 120 may include an Ethernet interface, an optical interface, a coaxial interface, an infrared interface, a radio frequency (RF) interface, a universal serial bus (USB) interface, a Wi-Fi interface, a cellular network interface, or the like.

The number and arrangement of the components of the ultrasound system 100 shown in FIG. 1 are provided as an example. In practice, the ultrasound system 100 may include additional components, fewer components, different components, or differently arranged components than those shown in FIG. 1. Additionally, or alternatively, a set of components (e.g., one or more components) of the ultrasound system 100 may perform one or more functions described as being performed by another set of components of the ultrasound system 100.

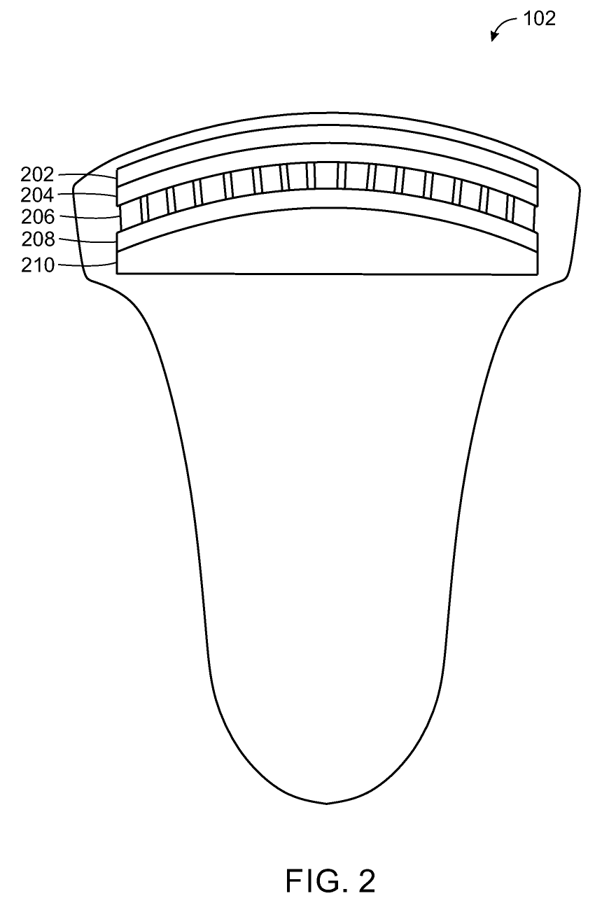

FIG. 2 is a diagram of an example ultrasound probe 102 of the ultrasound system 100 including one or more transducer elements 206 formed of a textured lead-based relaxor ferroelectric material fabricated using a single-crystal formed of a lead-based relaxor ferroelectric material, matrix particles formed of the lead-based relaxor ferroelectric material, and template particles formed of the lead-based relaxor ferroelectric material. As shown in FIG. 2, the ultrasound probe 102 may include a lens 202, an acoustic matching layer 204, transducer elements 206, an acoustic dematching layer 208, and a backing layer 210.

According to an embodiment, the lens 202 may be configured to direct an ultrasound signal towards the region of interest of the subject. For example, the lens 202 may be silicone, epoxy, rubber, or the like. According to an embodiment, the acoustic matching layer 204 may be configured to facilitate matching of an impedance differential that may exist between the relatively high impedance transducer elements 206 and the relatively low impedance subject. For example, the acoustic matching layer 204 may be graphite, plastic, resin, or the like. According to an embodiment, the transducer elements 206, respectively, may be configured to receive an element specific transmit signal, deform based on the element specific transmit signal, generate an ultrasound signal based on the deformation, and transmit the ultrasound signal towards a region of interest. Additionally, or alternatively, the transducer elements 206 may be configured to receive an echo signal reflected by or backscattered from the region of interest, deform based on the echo signal, generate an electrical signal based on the deformation, and transmit the electrical signal. For example, the transducer elements 206 may be piezoelectric materials, such as Pb(Mg_{1/3}Nb_{2/3})O₃-PbTiO₃ (“PMN-PT”), Pb(In_{1/2}Nb_{1/2})O₃- Pb(Mg_{1/3}Nb_{2/3})O₃-PbTiO₃ (“PIN-PMN-PT”), Pb(ZrTi) (“PZT”), or the like. According to an embodiment, the acoustic dematching layer 208 may be configured to decrease insertion losses and enhance a frequency bandwidth of the transducer elements 206. For example, the acoustic dematching layer 208 may be tungsten carbide, silicon carbide, or the like. According to an embodiment, the backing layer 210 may be configured to attenuate ultrasound signals directed from the transducer elements 206 in a direction opposite to the subject, and attenuate ultrasound signals deflected by a housing of the ultrasound probe 102. For example, the backing layer 210 may be an epoxy, a metal, or the like.

The number and arrangement of the components of the ultrasound probe 102 shown in FIG. 2 are provided as an example. In practice, the ultrasound probe 102 may include additional components, fewer components, different components, or differently arranged components than those shown in FIG. 2. Additionally, or alternatively, a set of components (e.g., one or more components) of the ultrasound probe 102 may perform one or more functions described as being performed by another set of components of the ultrasound probe 102.

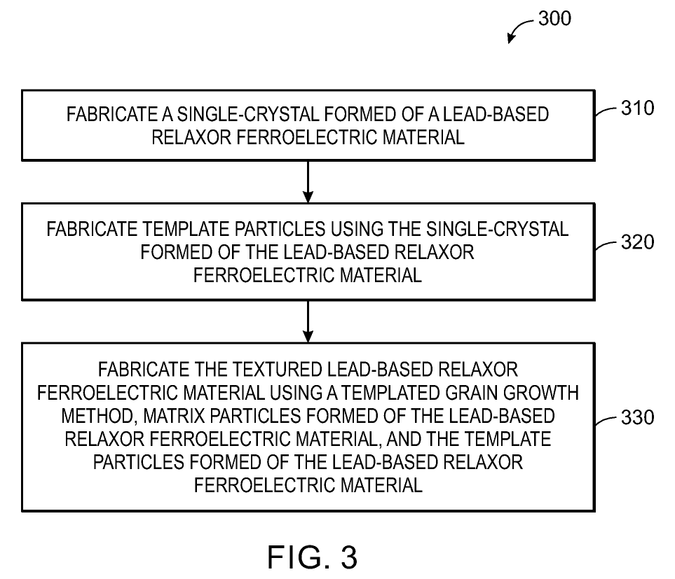

FIG. 3 is a flowchart of an example process for fabricating a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material.

As shown in FIG. 3, the process 300 may include fabricating a single-crystal formed of a lead-based relaxor ferroelectric material (operation 310). According to an embodiment, the single-crystal may be formed of PMN-PT material in the event that the textured lead-based relaxor ferroelectric material to be fabricated is PMN-PT material. Alternatively, the single-crystal may be formed of PIN-PMN-PT material in the event that the textured lead-based relaxor ferroelectric material to be fabricated is PIN-PMN-PT material. The single-crystal may be a material in which a crystal lattice of the entire material is continuous and unbroken to the edges of the material, and might not include any grain boundaries. According to an embodiment, the single-crystal may be a boule or an ingot, and may have a circumference of three inches, four inches, six inches, etc., and may have a length of ten inches, twelve inches, thirty inches, etc. According to an embodiment, the single-crystal formed of the lead-based relaxor ferroelectric material may be fabricated using a single-crystal growing process. For example, the single-crystal formed of the lead-based relaxor ferroelectric material may be fabricated using the Bridgman growth method, a solid-state crystal growth method, a flux method, the Czochralski method, the Vernuil method, or the like.

As further shown in FIG. 3, the process 300 may include fabricating template particles using the single-crystal formed of the lead-based relaxor ferroelectric material (operation 320). The template particles may be particles formed of the lead-based relaxor ferroelectric material. For example, the template particles may be formed of PMN-PT material in the event that the textured lead-based relaxor ferroelectric material to be fabricated is PMN-PT material. Alternatively, the template particles may be PIN-PMN-PT material in the event that the textured lead-based relaxor ferroelectric material to be fabricated is PIN-PMN-PT material. The template particles may be anisotropic. For example, the template particles may have a thickness of 0.25 µm, 0.50 µm, 0.75 µm, etc. Further, the template particles may have a width of 1 µm, 2 µm, 10 µm, 20 µm, etc. Further, the template particles may have a length of 2 µm, 5 µm, 10 µm, 20 µm, etc.

According to an embodiment, the template particles may be fabricated by cutting, slicing, crushing, milling, dicing, etc., the single-crystal formed of the lead-based relaxor ferroelectric material. For example, the single-crystal formed of the lead-based relaxor ferroelectric material may be cut along a longitudinal axis of the single-crystal to form a set of wafers. Each wafer may have a circumference that corresponds to the circumference of the single-crystal. For example, each wafer may have a circumference of three inches, four inches, six inches, etc. Each wafer may have a thickness that is less than the length of the original single-crystal before the cutting of the single-crystal. For example, each wafer may have a thickness of 0.25 µm, 0.50 µm, 0.75 µm, etc. In this way, a singe-crystal may be processed to form multiple wafers. A wafer may then be processed to form a set of template particles. For example, a wafer may be cut to form a set of template particles that each have a particular set of dimensions.

As further shown in FIG. 3, the process 300 may include fabricating the textured lead-based relaxor ferroelectric material using a templated grain growth method, matrix particles formed of the lead-based relaxor ferroelectric material, and the template particles formed of the lead-based relaxor ferroelectric material (operation 330). According to an embodiment, the templated grain growth method may include orienting the template particles in the matrix particles. For example, the orienting the template particles in the matrix particles may include a tape casting method, a slip casting method, a hot pressing method, or the like. In this way, the larger template particles are dispersed and oriented in the matrix particles which are relatively smaller and equiaxed particles. The matrix particles may be in a powder form, a slurry form, or the like. One or more additives may be added to the matrix particles. For example, the additives may include binders, resins, or the like. After being oriented in the matrix particles, the templated grain growth method may include sintering the combination of the template particles and the matrix particles to form a solid mass of the textured lead-based relaxor ferroelectric material.

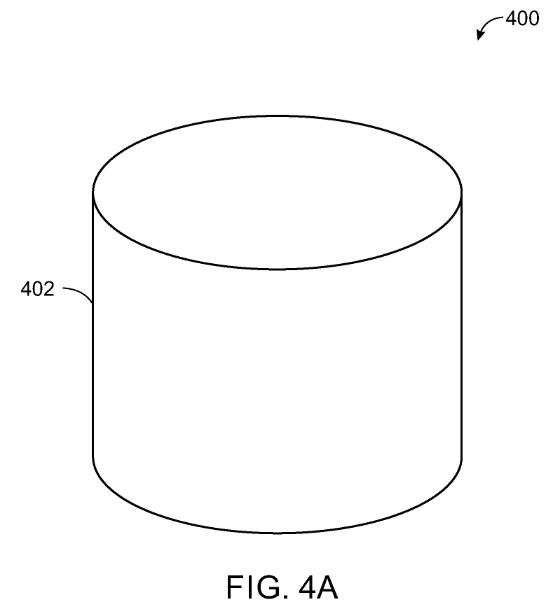

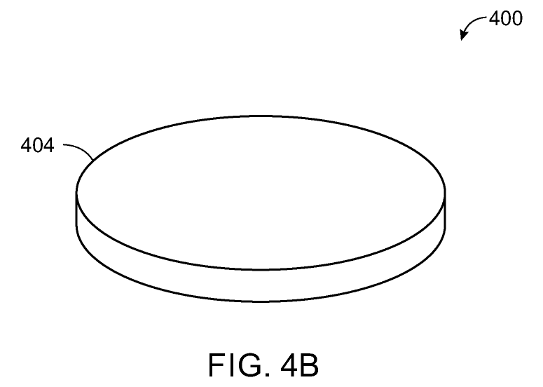

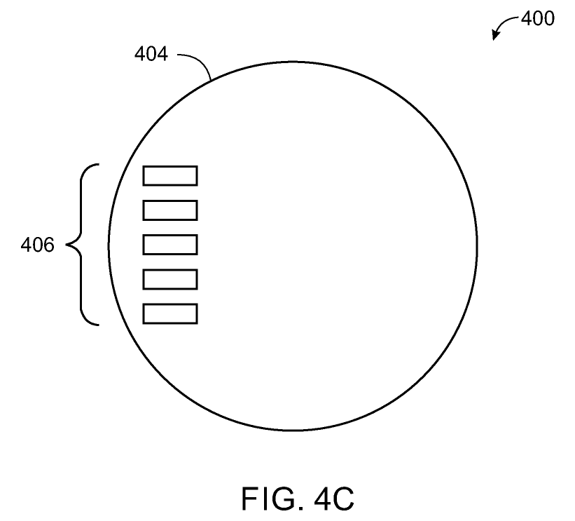

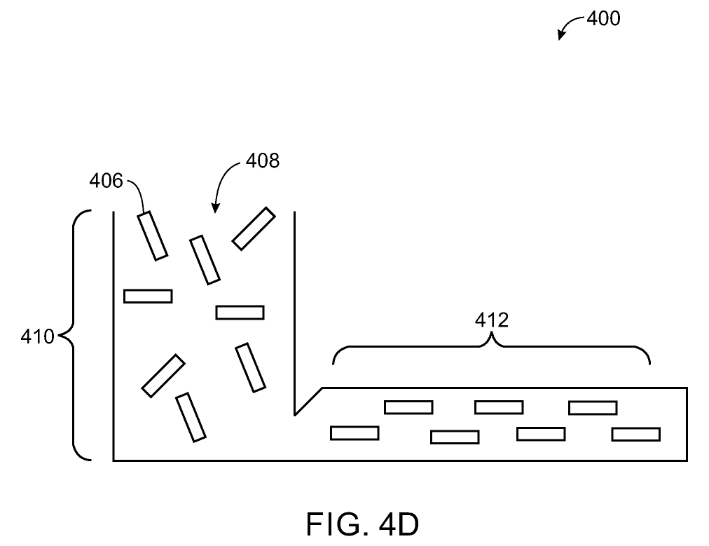

FIGS. 4A-4D are diagrams of a process 400 for fabricating a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material. As shown in FIG. 4A, an ingot 402 of a single-crystal formed of a lead-based relaxor ferroelectric material may have a substantially cylindrical shape and corresponding dimensions. As shown in FIG. 4B, the ingot 402 may be processed to obtain a wafer 404 of the single-crystal formed of a lead-based relaxor ferroelectric material. It should be understood that multiple wafers 404 may be obtained based on the ingot 402 by cutting the ingot 402 along a longitudinal axis of the ingot 402. As shown in FIG. 4C, the wafer 404 may be processed to obtain template particles 406 formed of the lead-based relaxor ferroelectric material. For instance, as shown, the template particles 406 may be cut from the wafer 404, and may have substantially rectangular shapes and corresponding dimensions. It should be understood that multiple template particles 406 may be obtained from the wafer 404. In some embodiments, the wafer 404 may be processed to obtain a single-crystal piezoelectric transducer element, and that the wafer 404 may be further processed to obtain template particles 406 using vestigial portions of the wafer 404. As shown in FIG. 4D, the template particles 406 may be added to matrix particles 408 formed of the lead-based relaxor ferroelectric material. In a mixture 410 of the template particles 406 and the matrix particles 408, the template particles 406 may be substantially unaligned. In a mixture 412 of the template particles 406 and the matrix particles 408, the template particles 406 may be oriented using a tape casting method. After being oriented, the mixture 412 may be sintered to obtain a textured lead-based relaxor ferroelectric material.

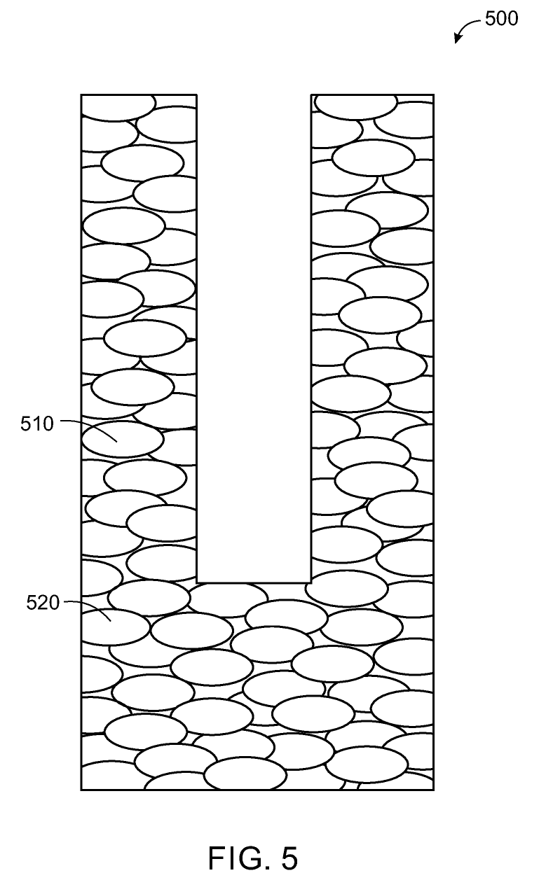

FIG. 5 is a diagram 500 of a textured lead-based relaxor ferroelectric material fabricated using a single-crystal formed of a lead-based relaxor ferroelectric material, matrix particles formed of the lead-based relaxor ferroelectric material, and template particles formed of the lead-based relaxor ferroelectric material. As shown in FIG. 5, the textured lead-based relaxor ferroelectric material may include template particles 510 formed of a lead-based relaxor ferroelectric material and matrix particles 520 formed of the same underlying lead-based relaxor ferroelectric material. The textured lead-based relaxor ferroelectric material may have improved mechanical stability after dicing and high homogeneity.

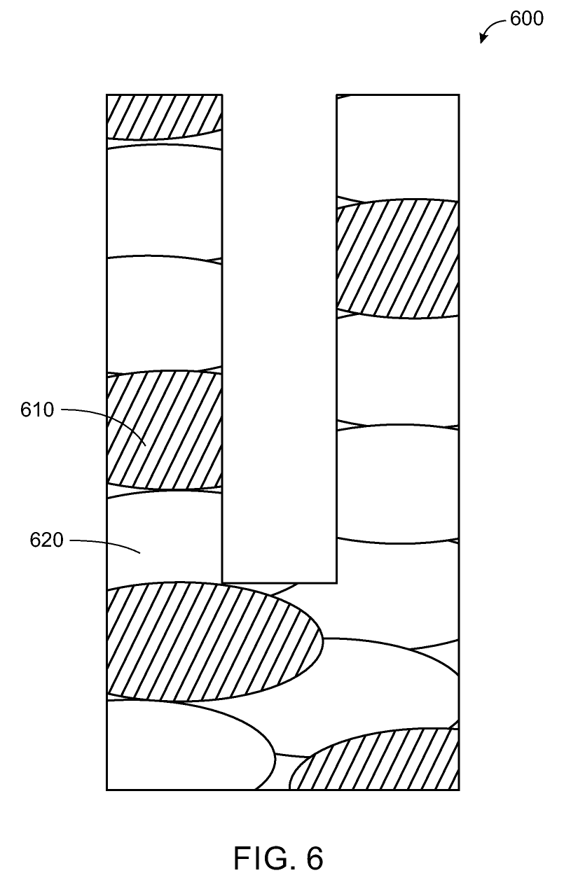

FIG. 6 is a diagram 600 of a textured lead-based relaxor ferroelectric material fabricated using matrix particles formed of a lead-based relaxor ferroelectric material, and template particles formed of barium titanate material. As shown in FIG. 6, the textured lead-based relaxor ferroelectric material may include template particles 610 formed of barium titanate material, and matrix particles 620 formed of a lead-based relaxor ferroelectric material. The textured lead-based relaxor ferroelectric material may have a lack of mechanical stability after dicing and poor homogeneity.

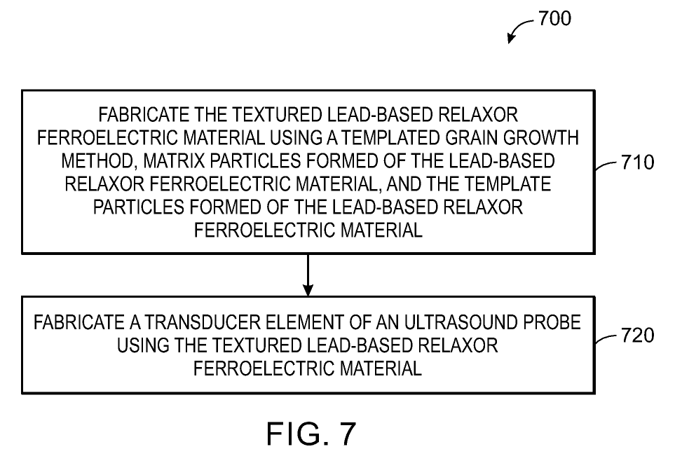

FIG. 7 is a flowchart of an example process 700 for fabricating a transducer element of an ultrasound probe using a textured lead-based relaxor ferroelectric material fabricated using a templated grain growth method, a single-crystal formed of a lead-based relaxor ferroelectric material, matrix particles formed of the lead-based relaxor ferroelectric material, and template particles formed of the lead-based relaxor ferroelectric material.

As shown in FIG. 7, the process 700 may include fabricating a textured lead-based relaxor ferroelectric material using a templated grain growth method, matrix particles formed of the lead-based relaxor ferroelectric material, and the template particles formed of the lead-based relaxor ferroelectric material (operation 710). For example, the operation 710 may include substantially similar operations as described in connection with FIG. 3.

As further shown in FIG. 7, the process 700 may include fabricating a transducer element of an ultrasound probe using the textured lead-based relaxor ferroelectric material (operation 720). For example, the textured lead-based relaxor ferroelectric material may be used to fabricate a transducer element of an ultrasound probe. Further, the transducer element may be used to fabricate an ultrasound probe.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A textured lead-based relaxor ferroelectric material for a transducer element of an ultrasound probe, the textured lead-based relaxor ferroelectric material comprising:
matrix particles formed of a lead-based relaxor ferroelectric material; and
template particles formed of the lead-based relaxor ferroelectric material.

2. The textured lead-based relaxor ferroelectric material for the transducer element of the ultrasound probe of claim 1, wherein the lead-based relaxor ferroelectric material is lead magnesium niobate-lead titanate (“PMN-PT”).

3. The textured lead-based relaxor ferroelectric material for the transducer element of the ultrasound probe of claim 1, wherein the lead-based relaxor ferroelectric material is lead indium niobate-lead magnesium niobate-lead titanate (“PIN-PMN-PT”).

4. The textured lead-based relaxor ferroelectric material for the transducer element of the ultrasound probe of claim 1, wherein the template particles are fabricated using a single-crystal formed of the lead-based relaxor ferroelectric material.

5. The textured lead-based relaxor ferroelectric material for the transducer element of the ultrasound probe of claim 1, wherein the textured lead-based relaxor ferroelectric material is fabricated using a templated grain growth method, the matrix particles, and the template particles.

6. The textured lead-based relaxor ferroelectric material for the transducer element of the ultrasound probe of claim 5, wherein the templated grain growth method includes orienting the template particles in the matrix particles.

7. The textured lead-based relaxor ferroelectric material for the transducer element of the ultrasound probe of claim 6, wherein the templated grain growth method includes sintering the template particles and the matrix particles after orienting the template particles in the matrix particles.

8. A method of fabricating a textured lead-based relaxor ferroelectric material for a transducer element of an ultrasound probe, the method comprising:
fabricating a single-crystal formed of a lead-based relaxor ferroelectric material;
fabricating template particles using the single-crystal formed of the lead-based relaxor ferroelectric material; and
fabricating the textured lead-based relaxor ferroelectric material using a templated grain growth method, matrix particles formed of the lead-based relaxor ferroelectric material, and the template particles formed of the lead-based relaxor ferroelectric material.

9. The method of fabricating the textured lead-based relaxor ferroelectric material for the transducer element of the ultrasound probe of claim 8, wherein the lead-based relaxor ferroelectric material is lead magnesium niobate-lead titanate (“PMN-PT”).

10. The method of fabricating the textured lead-based relaxor ferroelectric material for the transducer element of the ultrasound probe of claim 8, wherein the lead-based relaxor ferroelectric material is lead indium niobate-lead magnesium niobate-lead titanate (“PIN-PMN-PT”).

11. The method of fabricating the textured lead-based relaxor ferroelectric material for the transducer element of the ultrasound probe of claim 8, wherein the fabricating the single-crystal formed of the lead-based relaxor ferroelectric material includes a Bridgman growth method, a solid-state crystal growth method, or a flux method.

12. The method of fabricating the textured lead-based relaxor ferroelectric material for the transducer element of the ultrasound probe of claim 8, wherein the templated grain growth method includes orienting the template particles in the matrix particles.

13. The method of fabricating the textured lead-based relaxor ferroelectric material for the transducer element of the ultrasound probe of claim 12, wherein the orienting the template particles in the matrix particles includes a tape casting method, a slip casting method, or a hot pressing method.

14. The method of fabricating the textured lead-based relaxor ferroelectric material for the transducer element of the ultrasound probe of claim 8, wherein the templated grain growth method includes sintering the template particles and the matrix particles after orienting the template particles in the matrix particles.

15. A textured lead-based relaxor ferroelectric material for a transducer element of an ultrasound probe, the textured lead-based relaxor ferroelectric material being fabricated by a method comprising:
fabricating a single-crystal formed of a lead-based relaxor ferroelectric material;
fabricating template particles using the single-crystal formed of the lead-based relaxor ferroelectric material; and
fabricating the textured lead-based relaxor ferroelectric material using a templated grain growth method, matrix particles formed of the lead-based relaxor ferroelectric material, and the template particles formed of the lead-based relaxor ferroelectric material.

16. The textured lead-based relaxor ferroelectric material for the transducer element of the ultrasound probe of claim 15, wherein the lead-based relaxor ferroelectric material is lead magnesium niobate-lead titanate (“PMN-PT”).

17. The textured lead-based relaxor ferroelectric material for the transducer element of the ultrasound probe of claim 15, wherein the lead-based relaxor ferroelectric material is lead indium niobate-lead magnesium niobate-lead titanate (“PIN-PMN-PT”).

18. The textured lead-based relaxor ferroelectric material for the transducer element of the ultrasound probe of claim 15, wherein the fabricating the single-crystal formed of the lead-based relaxor ferroelectric material includes a Bridgman growth method, a solid-state crystal growth method, or a flux method.

19. The textured lead-based relaxor ferroelectric material for the transducer element of the ultrasound probe of claim 15, wherein the templated grain growth method includes orienting the template particles in the matrix particles.

20. The textured lead-based relaxor ferroelectric material for the transducer element of the ultrasound probe of claim 19, wherein the orienting the template particles in the matrix particles includes a tape casting method, a slip casting method, or a hot pressing method.
